SEQUENCE LISTING (33 sequences)

SEQ ID NO: 1 - DNA, 79 bp, Carica papaya
catgcatgccaatatcgataatcattcctcataactattacgttataagtaaaataactaaacaattacgttagttcat

SEQ ID NO: 2 - DNA, 67 bp, Carica papaya
ttaatgagaagaccatttgtcaagaaaattgtatgttgttcgaagattacattaacaagagagatgc

SEQ ID NO: 3 - DNA, 87 bp, Carica papaya
catcttgtatcataggttgtgctggcgttttcatgcctttgttgtgttctgtgtttggttgcaaattatataagtaacatttcaggt

SEQ ID NO: 4 - DNA, 80 bp, Carica papaya
taggtataaatggtgtaggtaaatcaatttttgtactaccaccactttttttcgcaatgatgataaataggggtcagtcc

SEQ ID NO: 5 - DNA, 58 bp, Carica papaya
ttagcatcaagtcctcaagagagaaagggaatccaagaatccaaggtagtttggccaa

SEQ ID NO: 6 - DNA, 90 bp, Carica papaya
gatcggtctgaaactcttgatattattggccctaatgatttttgggataaaaatcataaaagtagagttgatgaatttaataaaagaagc

SEQ ID NO: 7 - DNA, 90 bp, Carica papaya
agaagaggcaccattcaccaagatagcaaaagatgacataaaaatacataccctaatccatttcctccacttctctccaaaccaagtctc

SEQ ID NO: 8 - DNA, 81 bp, Carica papaya
tgacaataggtgagatggaagcaaaaaatatatatctaccaaagatctggcacacatggcatgtctaaagcttgagctggc

SEQ ID NO: 9 - DNA, 67 bp, Carica papaya
ctttctctttcaggtatcagtcaaaaagttctctctaaacctacctcaaatcacttccctagcgttt

SEQ ID NO: 10 - DNA, 66 bp, Carica papaya
aagatcacgcaggaaagagacctggtagaagaagagcccgcggggagaggtatccgagaggaagaa

SEQ ID NO: 11 - DNA, 79 bp, Carica papaya
caatggaagggactagaagatagtaaagaccacctggaaagatactcggtatttaaagtagcagttccctcatataaac

SEQ ID NO: 12 - DNA, 18 bp, Artificial Sequence
  Synthetic
catgcatgccaatatcga

SEQ ID NO: 13 - DNA, 26 bp, Artificial Sequence
  Synthetic
atgaactaacgtaattgtttagttat

SEQ ID NO: 14 - DNA, 24 bp, Artificial Sequence
  Synthetic
ttaatgagaagaccatttgtcaag

SEQ ID NO: 15 - DNA, 25 bp, Artificial Sequence
  Synthetic
gcatctctcttgttaatgtaatctt

SEQ ID NO: 16 - DNA, 22 bp, Artificial Sequence
  Synthetic
catcttgtatcataggttgtgc

SEQ ID NO: 17 - DNA, 27 bp, Artificial Sequence
  Synthetic
acctgaaatgttacttatataatttgc

SEQ ID NO: 18 - DNA, 26 bp, Artificial Sequence
  Synthetic
taggtataaatggtgtaggtaaatca

SEQ ID NO: 19 - DNA, 22 bp, Artificial Sequence
  Synthetic
ggactgacccctatttatcatc

SEQ ID NO: 20 - DNA, 21 bp, Artificial Sequence
  Synthetic
ttagcatcaagtcctcaagag

SEQ ID NO: 21 - DNA, 18 bp, Artificial Sequence
  Synthetic
ttggccaaactaccttgg

SEQ ID NO: 22 - DNA, 20 bp, Artificial Sequence
  Synthetic
gatcggtctgaaactcttga

SEQ ID NO: 23 - DNA, 27 bp, Artificial Sequence
  Synthetic
gcttcttttattaaattcatcaactct

SEQ ID NO: 24 - DNA, 18 bp, Artificial Sequence
  Synthetic
agaagaggcaccattcac

SEQ ID NO: 25 - DNA, 20 bp, Artificial Sequence
  Synthetic
gagacttggtttggagagaa

SEQ ID NO: 26 - DNA, 21 bp, Artificial Sequence
  Synthetic
tgacaataggtgagatggaag

SEQ ID NO: 27 - DNA, 18 bp, Artificial Sequence
  Synthetic
gccagctcaagctttaga

SEQ ID NO: 28 - DNA, 23 bp, Artificial Sequence
  Synthetic
ctttctctttcaggtatcagtca

SEQ ID NO: 29 - DNA, 20 bp, Artificial Sequence
  Synthetic
aaacgctagggaagtgattt

SEQ ID NO: 30 - DNA, 19 bp, Artificial Sequence
  Synthetic
aagatcacgcaggaaagag

SEQ ID NO: 31 - DNA, 20 bp, Artificial Sequence
  Synthetic
tttcttcctctcggatacct

SEQ ID NO: 32 - DNA, 23 bp, Artificial Sequence
  Synthetic
caatggaagggactagaagatag

SEQ ID NO: 33 - DNA, 23 bp, Artificial Sequence
  Synthetic
gtttatatgagggaactgctact

(19) United States
(12) Patent Application Publication
(10) Pub. No.: US 2021/0180142 A1
(43) Pub. Date: Jun. 17, 2021

(54) Molecular Markers Associated with Fruiting of Papaya

(71) Applicant: NATIONAL PINGTUNG UNIVERSITY OF SCIENCE AND TECHNOLOGY, NEIPU (TW)

(72) Inventors: Chen-Yu Lee, Neipu (TW); Fure-Chyi Chen, Neipu (TW); Shih-Wen Chin, Neipu (TW); Ting-Chi Cheng, Neipu (TW)

(21) Appl. No.: 16/716,288

(22) Filed: Dec. 16, 2019

(30) Foreign Application Priority Data
Dec. 12, 2019 (TW) 108145572

(51) Int. Cl. C12Q 1/6895; C12Q 1/6869; C12Q 1/6853

(52) U.S. Cl. CPC C12Q 1/6895; C12Q 2600/158; C12Q 1/6853; C12Q 1/6869

(57) ABSTRACT

The present disclosure provides a molecular marker for identifying fruiting characteristics of hermaphroditic papayas, wherein the molecular marker is selected from any of the nucleotide sequence group of SEQ ID NO: 1 to 11. This present disclosure also provides a method of using molecular markers for identifying fruiting characteristics of hermaphroditic papaya.

### CROSS-REFERENCE TO RELATED APPLICATIONS

This application claims priority to Taiwanese application No. 108145572 having a filing date of Dec. 12, 2019, the entire contents of which is hereby incorporated by reference.

### REFERENCE TO SEQUENCE LISTING

This application contains a Sequence Listing submitted as an electronic text file named “19-2371-US Sequence Listing Updated ST25.txt” having a size in bytes of 7 kb and originally created on Feb. 24, 2020. The information contained in this electronic file is hereby incorporated by reference in its entirety pursuant to 37 CFR § 1.52(e)(5).

### FIELD OF TECHNOLOGY

The present disclosure provides a molecular marker associated with the fruiting characteristics of papaya and its use. More specifically, the disclosure provides molecular markers and methods for identifying fruiting characteristics of hermaphroditic papayas.

### BACKGROUND

Papaya (Carica papaya L.) is a widely planted and important cash crop. It can be dioecious with a staminate plant or pistillate plant, or hermaphroditic with both staminate and carpellate parts on the same flower. When planting papayas, in view of lowering cost and increasing crop productivity, since hermaphroditic plants can self-pollinate and produce fruits, they are preferred over staminate plants that fail to produce fruits, or pistillate plants that require artificial pollination. Papaya seedling gender cannot be determined based on appearance. Therefore, in order to obtain hermaphroditic plants with more commercial value, significant time and effort must be invested in planting and cultivating until it is possible to differentiate the gender of the papaya plant by its floral organs.

Furthermore, the fruiting characteristics of hermaphroditic papaya are associated with the innate stability of the floral organs. Hermaphroditic papayas with stable floral organs continue to produce fruit during the fruiting period. Hermaphroditic papayas with unstable floral organs bloom but not fruit during the fruiting period due to degeneration of the floral organs by environmental factors such as temperature and/or soil nutrition. Hermaphroditic papayas with unstable fruiting characteristics may also be parthenocarpic and produce malformed fruits. Both cases result in harvest loss.

Traditionally, vegetative propagation methods such as tissue culture, cutting, and grafting were used to preserve and ensure the expected sexuality of papayas. However, these methods are associated with high cost and high technical threshold. Recently, in order to increase the efficiency of identifying the gender of papayas, molecular markers including RAPD, AFLP, RFLP, and SNP have been identified that provide a technical approach for identifying the gender of papaya seedlings when they cannot be distinguished by appearance. (Taiwan patent publication numbers 201823470 and 201825683). The aforementioned Taiwanese patent publications disclose a molecular marker that can be used to identify the gender of papayas. However, there is an absence of technical teachings on how to identify the fruiting characteristics of hermaphroditic papayas before they fruit thereby allowing for the selection of hermaphroditic papaya strains with stable fruiting characteristics (papayas that will fruit stably during fruiting season instead of producing malformed fruits). There are no technical teachings to sufficiently solve this issue to date.

### SUMMARY

The present disclosure provides a molecular marker and associated methods for identifying the fruiting characteristics of papayas before they fruit. Therefore, hermaphroditic papaya strains with stable fruiting characteristic can be selected.

The present disclosure, in one embodiment provides a molecular marker for identifying the fruiting characteristics of hermaphroditic papayas, wherein the molecular markers are selected from the nucleotides corresponding to SEQ ID NOs: 1 to 11. In another embodiment of the present disclosure is a probe for identifying the fruiting characteristics of hermaphroditic papayas, wherein the probe is used for detecting the molecular markers.

In yet another embodiment of the present disclosure is a kit for identifying the fruiting characteristics of hermaphroditic papayas, wherein the kit comprises the probe.

In still another embodiment of the present disclosure is a method for identifying the fruiting characteristics of hermaphroditic papayas, wherein the method comprises obtaining genomic DNA from hermaphroditic papayas and analyzing whether the genomic DNA comprises one or more of the nucleotide sequences of group (i) comprising: SEQ ID NO: 3, SEQ ID NO: 5, SEQ ID NO: 10, or SEQ ID NO: 11; or group (ii) comprising SEQ ID NO: 9; wherein the existence of at least one nucleotide sequence of the group (i) indicates an unstable fruiting characteristic of the hermaphroditic papayas, further wherein the unstable fruiting characteristic is a lack of fruiting during the fruiting period or fruiting of malformed fruits; or wherein the existence of the nucleotide sequence of group (ii) indicates a stable fruiting characteristic of hermaphroditic papaya, further wherein the stable fruiting characteristics is fruiting performance without fruit absence during the fruiting period or without fruiting of malformed fruits.

In yet another embodiment of the present disclosure is a high-resolution melting analysis; wherein the high resolution melting analysis detects one of the following SEQ ID NOs: using one of the following primer pair groups: (a) primer pair SEQ ID NO: 16 and SEQ ID NO: 17 for detecting SEQ ID NO: 3; (b) primer pair SEQ ID NO: 20 and SEQ ID NO: 21 for detecting SEQ ID NO: 5 (c) primer pair SEQ ID NO: 30 and SEQ ID NO: 31 for detecting SEQ ID NO: 10; (d) primer pair SEQ ID NO: 32 and SEQ ID NO: 33 for detecting SEQ ID NO: 11 or (e) primer pair SEQ ID NO: 28 and SEQ ID NO: 29 for detecting SEQ ID NO: 9.

In still another embodiment of the present disclosure is a method for identifying the fruiting characteristics of hermaphroditic papayas, comprising the steps of: obtaining genomic DNA from hermaphroditic papayas; analyzing the genomic DNA comprises a homozygous allele consisting of the nucleotide sequence of one of group (i) consisting of SEQ ID NO: 1, SEQ ID NO: 2, SEQ ID NO: 4 or group (ii) consisting of SEQ ID NO: 6, SEQ ID NO: 7, SEQ ID NO 8; wherein the existence of at least one homozygous allele consisting of the nucleotide sequences of group (i) indicates the fruiting characteristic of the hermaphroditic papayas is unstable; wherein the unstable fruiting characteristic is lacking fruiting during fruiting period or fruiting of malformed fruits; and further wherein the existence of at least one homozygous allele consisting of the nucleotide sequences of (ii) indicates the fruiting characteristic of the hermaphroditic papayas is stable; wherein the stable fruiting characteristics is fruiting performance without lacking fruiting during fruiting period nor fruiting of malformed fruits.

In a further embodiment of the present disclosure is a method wherein the analysis is a high resolution melting analysis; wherein the high resolution melting analysis uses at least one primer pair, group wherein the primer pair group and associated primers are group: (a) primer pair for detecting SEQ ID NO: 1, comprising SEQ ID NO: 12 and SEQ ID NO: 13; (b) primer pair for detecting SEQ ID NO: 2, comprising SEQ ID NO: 14 and SEQ ID NO: 15; (c) primer pair for detecting SEQ ID NO: 4, comprising SEQ ID NO: 18 and SEQ ID NO: 19; (d) primer pair for detecting SEQ ID NO: 6, comprising SEQ ID NO: 22 and SEQ ID NO: 23; (e) primer pair for detecting SEQ ID NO: 7, comprising SEQ ID NO: 24 and SEQ ID NO: 25; or (f) primer pair for detecting SEQ ID NO: 8, comprising SEQ ID NO: 26 and SEQ ID NO: 27.

In a further embodiment of the present disclosure is a high-resolution melting analysis kit for identifying the fruiting characteristics of hermaphroditic papayas, wherein the kit comprises one or more primer pairs selected from any of the following groups, wherein the primers are: (a) primer pair for detecting SEQ ID NO: 1, comprising SEQ ID NO: 12 and SEQ ID NO: 13; (b) primer pair for detecting SEQ ID NO: 2, comprising SEQ ID NO: 14 and SEQ ID NO: 15; (c) primer pair for detecting SEQ ID NO: 3, comprising SEQ ID NO: 16 and SEQ ID NO: 17; (d) primer pair for detecting SEQ ID NO: 4, comprising SEQ ID NO: 18 and SEQ ID NO: 19; (e) primer pair for detecting SEQ ID NO: 5, comprising SEQ ID NO: 20 and SEQ ID NO: 21; (f) primer pair for detecting SEQ ID NO: 6, comprising SEQ ID NO: 22 and SEQ ID NO: 23; (g) primer pair for detecting SEQ ID NO: 7, comprising SEQ ID NO: 24 and SEQ ID NO: 25; (h) primer pair for detecting SEQ ID NO: 8, comprising SEQ ID NO: 26 and SEQ ID NO: 27; (i) primer pair for detecting SEQ ID NO: 9, comprising SEQ ID NO: 28 and SEQ ID NO: 29; (j) primer pair for detecting SEQ ID NO: 10, comprising SEQ ID NO: 30 and SEQ ID NO: 31; or (k) primer pair for detecting SEQ ID NO: 11, comprising SEQ ID NO: 32 and SEQ ID NO: 33.

In a further embodiment of the present disclosure is a system for identifying the fruiting characteristics of hermaphroditic papayas, wherein the system is comprised of 11 molecular markers used for identifying fruiting characteristics of hermaphroditic papayas, wherein the 11 molecular markers used for identifying the fruiting characteristics of hermaphroditic papayas comprises nucleotide sequences SEQ ID NO: 1 to 11.

In one aspect is a recombinant vector comprising the aforementioned molecular markers for identifying the fruiting characteristics of hermaphroditic papayas.

Furthermore, the presentation disclosure also provides single nucleotide polymorphism (SNP) markers for identifying the fruiting characteristics of hermaphroditic papayas. The SNP markers are selected from the following groups:

SNP1: “A” at position 31 of SEQ ID NO: 1. If the hermaphroditic papaya expresses the SNP1 marker and the genotype at SNP1 locus is homozygous, the fruiting characteristic of the hermaphroditic papaya is unstable.

SNP2: “T” at position 29 of SEQ ID NO: 2. If the hermaphroditic papaya expresses the SNP2 marker and the genotype at SNP2 locus is homozygous, the fruiting characteristic of the hermaphroditic papaya is unstable.

SNP3: “G” at position 61 of SEQ ID NO: 3. If the hermaphroditic papaya expresses the SNP marker and regardless whether the genotype at SNP3 locus is heterozygous or homozygous, the fruiting characteristic of the hermaphroditic papaya is unstable.

SNP4: “T” at position 46 of SEQ ID NO: 4. If the hermaphroditic papaya has the SNP marker and the genotype at SNP4 locus is homozygous, the fruiting characteristic of the hermaphroditic papaya is unstable.

SNP5: “A” at position 24 of SEQ ID NO: 5. If the hermaphroditic papaya has the SNP marker and regardless whether the genotype at the SNP5 locus is heterozygous or homozygous, the fruiting characteristic of the hermaphroditic papaya is unstable.

SNP6: “T” at position 33 of SEQ ID NO: 6. If the hermaphroditic papaya under test has the SNP marker and the genotype at SNP6 locus is homozygous, the fruiting characteristic of the hermaphroditic papaya is stable.

SNP7: “T” at position 57 of SEQ ID NO: 6. If the hermaphroditic papaya has the SNP marker and the genotype at SNP7 locus is homozygous, the fruiting characteristic of the hermaphroditic papaya is stable.

SNP8: “T” at position 61 of SEQ ID NO: 7. If the hermaphroditic papaya has the SNP marker and the genotype at SNP8 locus is homozygous, the fruiting characteristic of the hermaphroditic papayas is stable.

SNP9: “A” at position 27 of SEQ ID NO: 8. If the hermaphroditic papaya has the SNP marker and the genotype at SNP9 locus is homozygous, the fruiting characteristic of the hermaphroditic papayas stable.

SNP10: “C” at position 51 of SEQ ID NO: 8. If the hermaphroditic papayas has the SNP marker and the genotype at SNP10 locus is homozygous, the fruiting characteristic of the hermaphroditic papaya is stable.

SNP11: “T” at position 42 of SEQ ID NO: 9. If the hermaphroditic papaya has the SNP marker and regardless if the genotype at SNP11 locus is heterozygous or homozygous, the fruiting characteristic of the hermaphroditic papaya is stable.

SNP12: “G” at position 24 of SEQ ID NO: 10. If the hermaphroditic papaya has the SNP marker and regardless if the genotype at SNP12 locus is heterozygous or homozygous, the fruiting characteristic of the hermaphroditic papaya is stable.

SNP13: “C” at position 30 of SEQ ID NO: 11. If the hermaphroditic papaya has the SNP marker and regardless whether the genotype at SNP13 locus is heterozygous or homozygous, the fruiting characteristic of the hermaphroditic papaya is unstable.

This present disclosure also provides a SNP marker probe for identifying the fruiting characteristics of hermaphroditic papayas, wherein the said SNP marker probe is used to detect the said SNP markers.

In a further embodiment of the present disclosure is a kit for identifying the fruiting characteristics of hermaphroditic papayas, wherein the kit is used to detect SNP markers.

The present disclosure allows fruiting characteristics of hermaphroditic papayas to be identified during early stages of development before the floral organs of hermaphroditic papaya have developed or initiated fruiting. Efficient, faster, and more accurate methods of selecting hermaphroditic papaya strains are provided herein. The selected papaya strains described herein comprise stable fruiting characteristics that do not lack fruit or produce malformed fruits during the fruiting period. Hence, the methods of the present disclosure can shorten the time period required for breeding species improvement in papaya strains, thereby, reducing the cost associated with papaya production.

### BRIEF DESCRIPTION OF THE DRAWINGS

FIG. 1 illustrates different fruiting characteristics of papayas; FIGS. 1A and 1D illustrate stable fruiting results; FIGS. 1B and 1E illustrate malformed fruits (circle); FIGS. 1C and 1F illustrate fruit lacking during the fruiting period (circle).

FIG. 2 illustrates a high-resolution melting curve using molecular marker S1 (SEQ ID NO:1);

FIG. 3 illustrates a high-resolution melting curve using molecular marker S2 (SEQ ID NO:2);

FIG. 4 illustrates a high-resolution melting curve using molecular marker S3 (SEQ ID NO:3);

FIG. 5 illustrates a high-resolution melting curve using molecular marker S4 (SEQ ID NO:4);

FIG. 6 illustrates a high-resolution melting curve using molecular marker S5 (SEQ ID NO:5);

FIG. 7 illustrates a high-resolution melting curve using molecular marker S6 (SEQ ID NO:6);

FIG. 8 illustrates a high-resolution melting curve using molecular marker S7 (SEQ ID NO:7);

FIG. 9 illustrates a high-resolution melting curve using molecular marker S8 (SEQ ID NO:8);

FIG. 10 illustrates a high-resolution melting curve using molecular marker S9 (SEQ ID NO:9);

FIG. 11 illustrates a high-resolution melting curve using molecular marker S10 (SEQ ID NO:10); and

FIG. 12 illustrates a high-resolution melting curve using molecular marker S11 (SEQ ID NO:11).

### DETAILED DESCRIPTION

Strains of hermaphroditic papayas and the papayas of the present disclosure comprise: Tainung No. 1, Tainung No. 2, HongFu, LuFu, SINTA, YuanYe, Tn6, Hong Kong, Eksotika2, Indonesian, Kuala Lumpur, Hawaiian Solo Sunset, Papaya Linda, Solo sunrise improve, Red Maradol, and Maradol Raja.

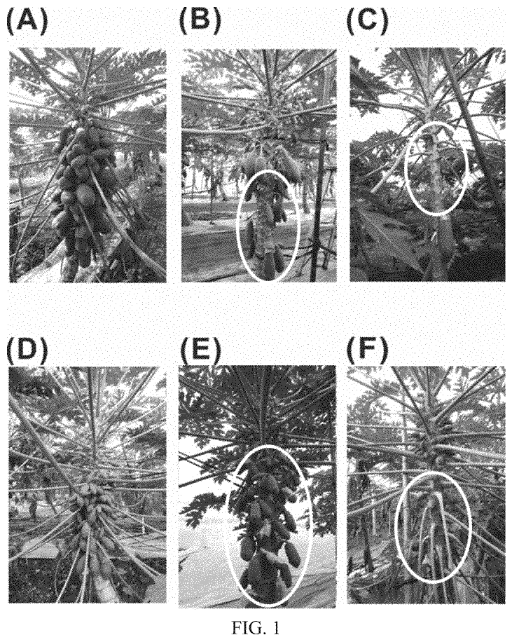

The term “fruiting characteristics” used herein indicates the fruiting performance of hermaphroditic papayas, including whether ripe papaya fruit enter the fruiting period, or whether the fruits are malformed. In the present disclosure, if the hermaphroditic papayas display “stable fruiting characteristics”, it can be predicted that the hermaphroditic papayas will neither lack fruit during fruiting period, nor will the fruits be malformed as depicted in FIGS. 1A and 1D. If the identification results of the hermaphroditic papayas indicate “unstable fruiting characteristics”, it can be predicted that the hermaphroditic papayas will lack fruit during the fruiting period as depicted in FIGS. 1C and 1F, or the fruits will be malformed.

The term “lacking fruit”, as used herein indicates that the flowering area of hermaphroditic papayas does not fruit during the fruiting period. This includes a lack of fruit in a portion, or all, of the flowering area.

The term “malformed fruits” as used herein indicates small or malformed hermaphroditic papaya fruits, which is caused by parthenocarpy, as indicated in by the circled areas in FIGS. 1B and 1E).

The term “alleles” as used herein indicates a sequence consisting of two or more variant sequence forms in genomic DNA or specific DNA chromosomes, causing the sequences to become mutual alleles. Variant sequence forms is used to indicate a mutation exists in one or more positions on the sequence. The term “homozygous alleles” as used herein indicates, the corresponding specific sequence of the allelosomal DNA only has one sequence form with no variation between the forms.

The term “probe” as used herein refers to substances that can be used to detect molecular markers. The probe can be an oligonucleotide that specifically hybridizes to a nucleotide sequence of the molecular marker. The oligonucleotide can further covalently bond chromophores and other molecules. The probe can include, but is not limited to, a PCR primer pair used to amplify the molecular marker nucleotide sequences of the present disclosure.

In the present disclosure, analysis of genomic DNA nucleotide sequences can be used to identify relative technical features known in art. Analysis techniques that may be applicable include, for example, Sanger's sequencing method, PCR-RFLP, TaqMan probe, High Resolution Melting (HRM), etc.

In the current disclosure, analysis of whether genomic DNA of hermaphroditic papayas express the target homozygous alleles can use relative technical features know in art. For example, wherein the oligonucleotide probe specifically hybridizes to the nucleotide sequence of homozygous alleles, High Resolution Melting (HRM), etc. may be applicable.

In a certain embodiment, “genomic DNA” of the present disclosure comprises the X chromosome DNA and the Yh chromosome DNA of papayas.

In one embodiment of the present disclosure, the kit can further comprise tools and/or reagents for collecting nucleic acid samples from hermaphroditic papayas. The kit of this present disclosure further comprises tools and/or reagents for preparing DNA from nucleic acid samples.

In one embodiment of the current disclosure, the kit can further comprise at least one primer pair, chosen from the following groups and, can be used to amplify nucleic acid sequence of the molecular markers in the present disclosure. The primers and associated molecular markers comprise:

(a) SEQ ID NO: 12 and SEQ ID NO: 13 for detecting SEQ ID NO: 1; (b) SEQ ID NO: 14 and SEQ ID NO: 15 for detecting SEQ ID NO: 2; (c) SEQ ID NO: 16 and SEQ ID NO: 17 for detecting SEQ ID NO: 3; (d) SEQ ID NO: 18 and SEQ ID NO: 19 for detecting SEQ ID NO: 4; (e) SEQ ID NO: 20 and SEQ ID NO: 21 for detecting SEQ ID NO: 5; (f) SEQ ID NO: 22 and SEQ ID NO: 23 for detecting SEQ ID NO: 6; (g) SEQ ID NO: 24 and SEQ ID NO: 25 for detecting SEQ ID NO: 7; (h) SEQ ID NO: 26 and SEQ ID NO: 27 for detecting SEQ ID NO: 8; (i) SEQ ID NO: 28 and SEQ ID NO: 29 for detecting SEQ ID NO: 9; (j) SEQ ID NO: 30 and SEQ ID NO: 31 for detecting SEQ ID NO: 10; or (k) SEQ ID NO: 32 and SEQ ID NO: 33 for detecting SEQ ID NO: 11.

In a one embodiment, the length of the probe used for identifying the fruiting characteristics of the hermaphroditic papaya has no special restrictions. The probe only needs to contain the nucleotide sequence of any of the molecular markers (SEQ ID NO: 1 to SEQ ID NO 11) of the present disclosure.

Examples 1-11 illustrate the application of 11 molecular markers in the present disclosure (nucleic sequence of SEQ ID NO: 1 to SEQ ID NO: 11, abbreviated S1-S11), which identify the hermaphroditic papaya samples. Results were obtained using high resolution fusion analyses.

### EXAMPLES

The Examples that follow are illustrative of specific embodiments of the disclosure, and various uses thereof. They are set forth for explanatory purposes only and should not be construed as limiting the scope of the disclosure in any way.

Genomic DNA was extracted from 12 hermaphroditic papayas with known fruiting characteristics, the numbers and known species name are listed in Table 1. Methods of extracting genomic DNA are technical teachings known by a person skilled in the art, hence it is not further described here.

TABLE 1
Hermaphroditic papaya characeristics

| | | | | Fruiting |
|---|---|---|---|---|
| No. | Origin | Species | Sexuality | Characteristics |
| 1 | Taiwan | Unconfirmed | Hermaphroditic | Unstable |
| 2 | East-West | SINTA | Hermaphroditic | Unstable |
| | Seed LTD.* | | | |
| 3 | Malaysia | HongKong | Hermaphroditic | Unstable |
| 4 | Taiwan | Unconfirmed | Hermaphroditic | Stable |
| 5 | Taiwan | Unconfirmed | Hermaphroditic | Stable |
| 6 | Hawaii | Hawaiian | Hermaphroditic | Unstable |
| | | Solo Sunset | | |
| 7 | Taiwan | Unconfirmed | Hermaphroditic | Unstable |
| 8 | Taiwan | Unconfirmed | Hermaphroditic | Stable |
| 9 | Taiwan | Unconfirmed | Hermaphroditic | Unstable |
| 10 | Taiwan | Unconfirmed | Hermaphroditic | Unstable |
| 11 | Taiwan | Unconfirmed | Hermaphroditic | Unstable |
| 12 | Taiwan | Unconfirmed | Hermaphroditic | Unstable |

*Thailand vegetable seed company

Primers (a)-(k) for the corresponding molecular markers in the present disclosure were used to analyze the 12 hermaphroditic papaya samples via high resolution melting analyses. Due to the differences of fruiting characteristics, each genomic DNA sample comprises at least one of the molecular maker nucleotide sequences of the present disclosure, or at least one homozygous allele of the nucleotide sequences of the molecular markers of the present disclosure. A single nucleotide sequence variance can cause difference in melting curve shape and location. Homozygous alleles and heterozygous alleles will also display different curve shape. Therefore, whether genomic DNA under test comprises the molecular marker of the present disclosure can be determined by the difference in curve shape, hence identifying the fruiting characteristics. Illustrative melting curve of each molecular markers are described respectively below.

### Example 1: Molecular Marker S1 (SEQ ID NO: 1)

When the genomic DNA of the hermaphroditic papaya comprises a homozygous allele pair, and the homozygous alleles comprise the nucleotide sequence of molecular marker S1 (SEQ ID NO: 1), the fruiting characteristics of the hermaphroditic papayas is unstable. When zero or one of the alleles of the hermaphroditic papayas comprises the nucleotide sequence of molecular marker S1, it indicates the fruiting characteristics of the hermaphroditic papaya is stable.

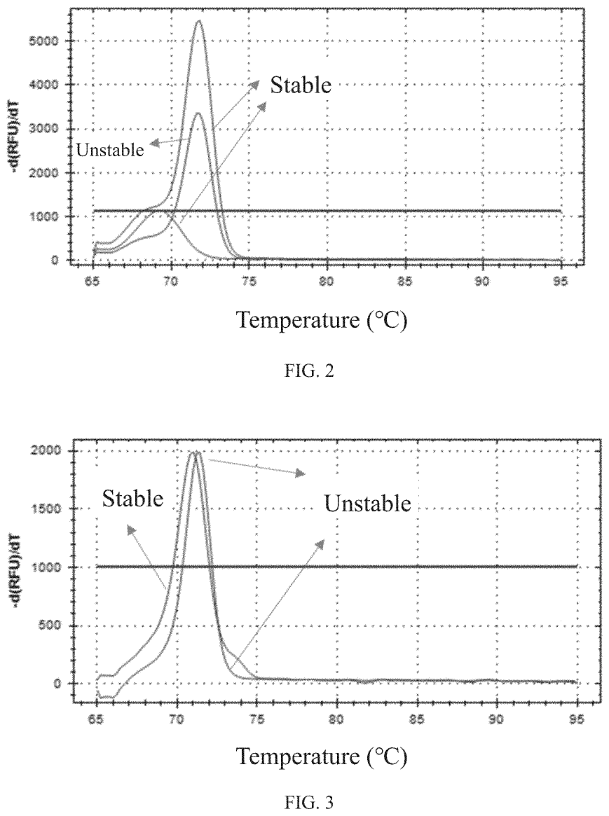

Primer pair (a)(SEQ ID NO: 12 and SEQ ID NO: 13) was used for HRM analysis. When the genomic DNA of hermaphroditic papayas comprise a homozygous allele pair of the nucleotide sequence of molecular marker S1, the curve shape and peak location of the melting curve will be similar to the “unstable” curve shown in FIG. 2. If zero or one allele comprises the nucleotide sequence of molecular marker S1, the curve shape and the peak location of the melting curve will be similar to the “stable” curve shown in FIG. 2.

### Example 2: Molecular marker S2 (SEQ ID NO: 2)

When the genomic DNA of the hermaphroditic papaya comprises a homozygous allele pair, and the homozygous alleles comprise the nucleotide sequence of molecular marker S2 (SEQ ID NO: 2), the fruiting characteristics of the hermaphroditic papaya is unstable. When zero or one allele of the hermaphroditic papaya comprise the nucleotide sequence of molecular marker S2, it indicates the fruiting characteristics of the hermaphroditic papayas is stable.

Primer pair (b)(SEQ ID NO: 14 and SEQ ID NO: 15) was used for HRM analysis. When the genomic DNA of hermaphroditic papayas comprise a homozygous allele pair of the nucleotide sequence of molecular marker S2, the curve shape and the peak location of the melting curve will be similar to the “unstable” curve shown in FIG. 3. If zero or one allele comprise the nucleotide sequence of the molecular marker S2, the curve shape and the peak location of the melting curve will be similar to the “stable” curve shown in FIG. 3.

### Example 3: Molecular marker S3 (SEQ ID NO:3)

When the genomic DNA of the hermaphroditic papaya comprises the nucleotide sequence of molecular marker S3 (SEQ ID NO: 3), the fruiting characteristics of the hermaphroditic papayas is unstable. When the hermaphroditic papayas does not express the nucleotide sequence of molecular marker S3, it indicates the fruiting characteristics of the hermaphroditic papaya is stable.

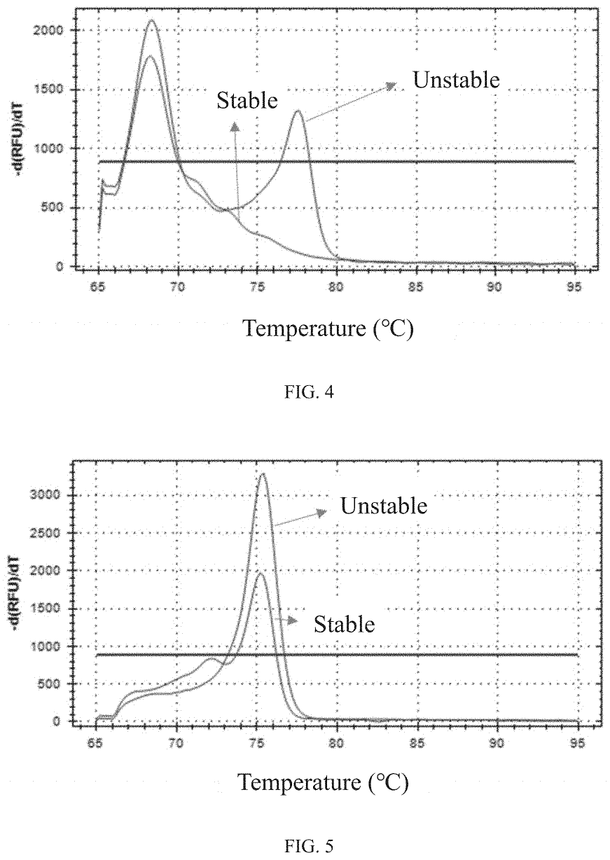

Primer pair (c)(SEQ ID NO: 16 and SEQ ID NO: 17) was used for HRM analysis. When the genomic DNA of hermaphroditic papayas comprise the nucleotide sequence of molecular marker S3, the curve shape and the peak location of the melting curve will be similar to the “unstable” curve shown in FIG. 4. If the papaya does not comprise nucleotide sequence of molecular marker S3, the curve shape and the peak location of the melting curve will be similar to the “stable” curve shown in FIG. 4.

### Example 4: Molecular marker S4 (SEQ ID NO: 4)

When the genomic DNA of the hermaphroditic papaya comprises a homozygous allele pair, and the homozygous alleles comprise the nucleotide sequence of molecular marker S4 (SEQ ID NO: 4), the fruiting characteristics of the hermaphroditic papaya is unstable. When zero or one allele of the hermaphroditic papaya expresses the nucleotide sequence of the molecular marker S4, it indicates the fruiting characteristics of the hermaphroditic papaya is stable.

Primer pair (d)(SEQ ID NO: 18 and SEQ ID NO: 19) was used for HRM analysis. When the genomic DNA of hermaphroditic papayas comprise a homozygous allele pair of the nucleotide sequence of the molecular marker S4, the curve shape and the peak location of the melting curve will be similar to the “unstable” curve shown in FIG. 5. If zero or one allele comprise the nucleotide sequence of molecular marker S4, the curve shape and the peak location of the melting curve will be similar to the “stable” curve shown in FIG. 5.

### Example 5: Molecular marker S5 (SEQ ID NO: 5)

When the genomic DNA of the hermaphroditic papaya comprises the nucleotide sequence of molecular marker S5 (SEQ ID NO: 5), the fruiting characteristics of the hermaphroditic papaya is unstable. When the hermaphroditic papaya does not comprise the nucleotide sequence of molecular marker S5, it indicates the fruiting characteristics of the hermaphroditic papaya is stable.

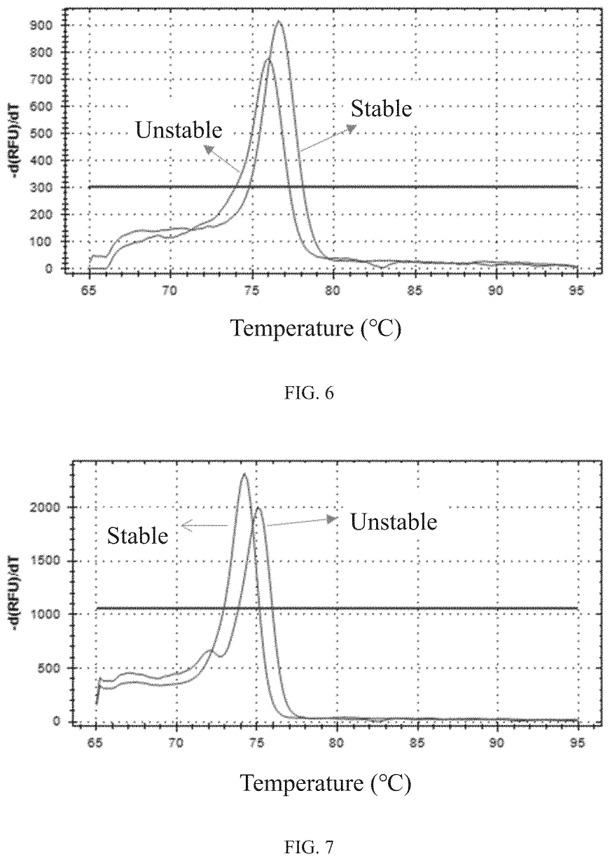

Primer pair (e)(SEQ ID NO: 20 and SEQ ID NO: 21) was used for HRM analysis. When the genomic DNA of hermaphroditic papayas comprise the nucleotide sequence of the molecular marker S5, the curve shape and the peak location of the melting curve will be similar to the “unstable” curve shown in FIG. 6. If the papaya does not comprise the nucleotide sequence of molecular marker S5, the curve shape and the peak location of the melting curve will be similar to the “stable” curve shown in FIG. 6.

### Example 6: Molecular marker S6 (SEQ ID NO: 6)

When the genomic DNA of the hermaphroditic papaya comprises a homozygous allele pair, and the homozygous alleles comprise the nucleotide sequence of molecular marker S6 (SEQ ID NO: 6), it indicates the fruiting characteristics of the hermaphroditic papaya is stable When zero or one allele of the hermaphroditic papaya comprises nucleotide sequence of the molecular marker S6, it indicates the fruiting characteristics of the hermaphroditic papaya is unstable.

Primer pair (f)(SEQ ID NO: 22 and SEQ ID NO: 23) was used for HRM analysis. When the genomic DNA of hermaphroditic papayas comprise a homozygous allele pair of the nucleotide sequence of the molecular marker S6, the curve shape and the peak location of the melting curve will be similar to the “stable” curve shown in FIG. 7. If zero or one allele comprises the nucleotide sequence of molecular marker S6, the curve shape and the peak location of the melting curve will be similar to the “unstable” curve shown in FIG. 7.

### Example 7: Molecular marker S7 (SEQ ID NO: 7)

When the genomic DNA of the hermaphroditic papaya comprises a homozygous allele pair, and the homozygous alleles comprise the nucleotide sequence of molecular marker S7 (SEQ ID NO: 7), the fruiting characteristics of the hermaphroditic papaya is stable. When zero or one allele of the hermaphroditic papaya comprises nucleotide sequence of molecular marker S7, it indicates the fruiting characteristics of the hermaphroditic papaya is unstable.

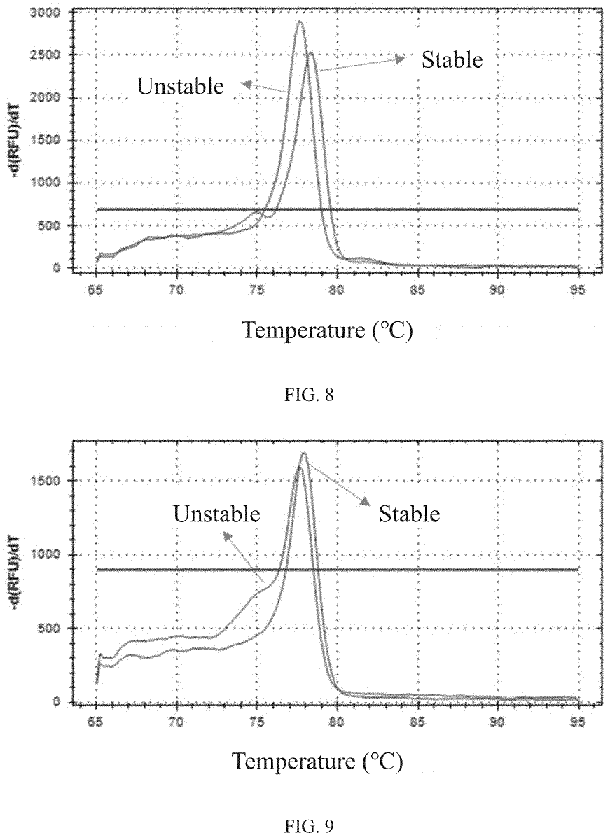

Primer pair (g)(SEQ ID NO: 24 and SEQ ID NO: 25) was used for HRM analysis. When the genomic DNA of hermaphroditic papayas comprise a homozygous allele pair of the nucleotide sequence of molecular marker S7, the curve shape and the peak location of the melting curve will be similar to the “stable” curve shown in FIG. 8. If zero or one allele comprises the nucleotide sequence of the molecular marker S7, the curve shape and the peak location of the melting curve will be similar to the “unstable” curve shown in FIG. 8.

### Example 8: Molecular marker S8 (SEQ ID NO: 8)

When the genomic DNA of the hermaphroditic papaya comprises a homozygous allele pair, and the homozygous alleles comprise the nucleotide sequence of molecular marker S8 (SEQ ID NO: 8), the fruiting characteristics of the hermaphroditic papaya is stable. When zero or one allele of the hermaphroditic papaya comprises nucleotide sequence of the molecular marker S8, the fruiting characteristics of the hermaphroditic papaya is unstable.

Primer pair (h)(SEQ ID NO: 26 and SEQ ID NO: 27) was used for HRM analysis. When the genomic DNA of hermaphroditic papayas comprise a homozygous allele pair of the nucleotide sequence of molecular marker S8, the curve shape and the peak location of the melting curve will be similar to the “stable” curve shown in FIG. 9. If zero or one allele comprises the nucleotide sequence of molecular marker S8, the curve shape and the peak location of the melting curve will be similar to the “unstable” curve shown in FIG. 9.

### Example 9: Molecular marker S9 (SEQ ID NO: 9)

When the genomic DNA of the hermaphroditic papaya comprises the nucleotide sequence of molecular marker S9 (SEQ ID NO: 9), the fruiting characteristics of the hermaphroditic papaya is stable. When the hermaphroditic papaya does not comprise the nucleotide sequence of molecular marker S9, the fruiting characteristics of the hermaphroditic papaya is unstable.

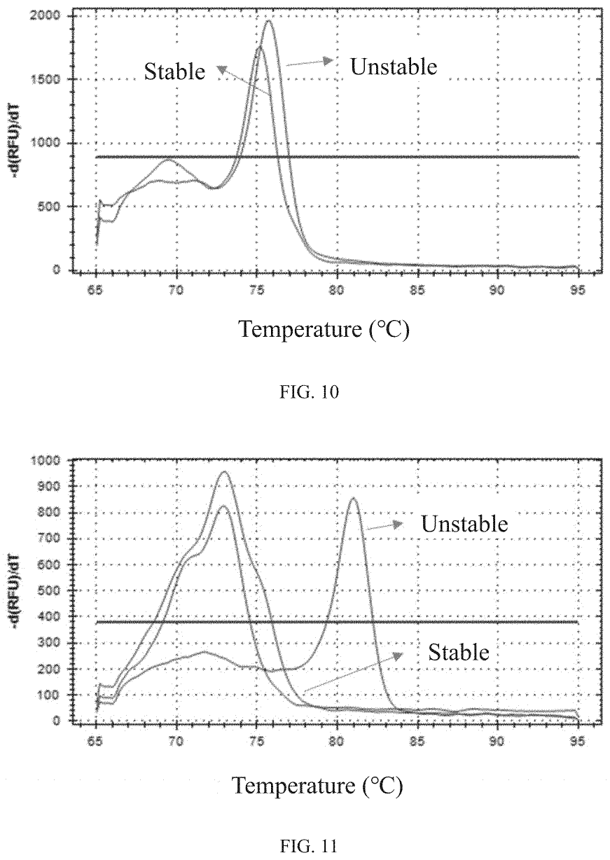

Primer pair (i)(SEQ ID NO: 28 and SEQ ID NO: 29) was used for HRM analysis. When the genomic DNA of hermaphroditic papayas comprise the nucleotide sequence of molecular marker S9, the curve shape and the peak location of the melting curve will be similar to the “stable” curve shown in FIG. 10. If the papaya does not comprise the nucleotide sequence of molecular marker S9, the curve shape and the peak location of the melting curve will be similar to the “unstable” curve shown in FIG. 10.

### Embodiment 10: Molecular marker S10 (SEQ ID NO: 10)

When the genomic DNA of the hermaphroditic papaya comprises the nucleotide sequence of molecular marker S10 (SEQ ID NO: 10), the fruiting characteristics of the hermaphroditic papaya is unstable. When the hermaphroditic papaya does not comprise the nucleotide sequence of molecular marker S10, it indicates the fruiting characteristics of the hermaphroditic papaya is stable.

Primer pair (j)(SEQ ID NO: 30 and SEQ ID NO: 31) was used for HRM analysis. When the genomic DNA of hermaphroditic papayas comprise the nucleotide sequence of molecular marker S10, the curve shape and the peak location of the melting curve will be similar to the “unstable” curve shown in FIG. 11. If the papaya does not comprise the nucleotide sequence of molecular marker S10, the curve shape and the peak location of the melting curve will be similar to the “stable” curve shown in FIG. 11.

### Example 11: Molecular marker S11 (SEQ ID NO: 11)

When the genomic DNA of the hermaphroditic papaya comprises the nucleotide sequence of molecular marker S11 (SEQ ID NO: 11) the fruiting characteristics of the hermaphroditic papayas under test is unstable. When the hermaphroditic papaya does not comprise the nucleotide sequence of molecular marker S11, the fruiting characteristics of the hermaphroditic papaya is stable.

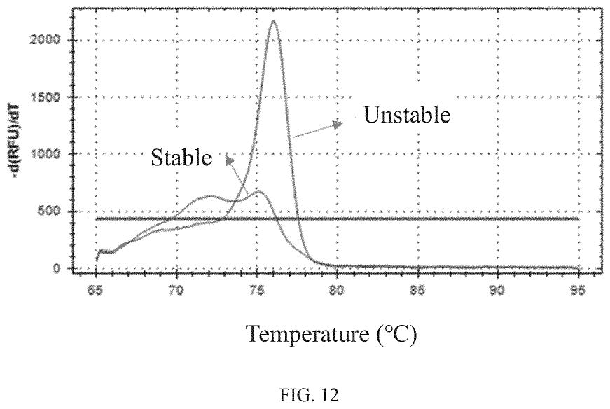

Primer pair (k)(SEQ ID NO: 32 and SEQ ID NO: 33) was used for HRM analysis. When the genomic DNA of hermaphroditic papayas comprise the nucleotide sequence of molecular marker S11, the curve shape and the peak location of the melting curve will be similar to the “unstable” curve shown in FIG. 12. If the papaya does not comprise the nucleotide sequence of molecular marker S11, the curve shape and the peak location of the melting curve will be similar to the “stable” curve shown in FIG. 12.

The embodiments above confirm that the molecular markers in the present disclosure can rapidly and accurately identify fruiting characteristics of different hermaphroditic papaya strains by using HRM analysis or other analysis methods disclosed in the embodiments. Through such identification in early stages of growth, hermaphroditic papayas with stable fruiting characteristics can be selected, hence reducing the chances of producing plants lacking fruit or fruiting malformed fruit.

Papayas are a cash crop, so the fruiting characteristic is an important breeding trait. The current disclosure, used in the papaya seedling industry can increase the breeding competitiveness of papaya by selecting hermaphroditic papaya strains with stable fruiting characteristics.

TABLE 2
Sequence Table

| Identifier | Sequence |
|---|---|
| SEQ ID NO: 1 | catgcatgccaatatcgataatcattcct |
| | cataactattacgttataagtaaaataac |
| | taaacaattacgttagttcat |
| SEQ ID NO: 2 | ttaatgagaagaccatttgtcaagaaaattgtat |
| | gttgttcgaagattacattaacaagagagatgc |
| SEQ ID NO: 3 | catcttgtatcataggttgtgctggcgttttcat |
| | gcctttgttgtgttctgtgtttggttgcaaatta |
| | tataagtaacatttcaggt |
| SEQ ID NO: 4 | taggtataaatggtgtaggtaaatcaatttttgt |
| | actaccaccactttttttcgcaatgatgataaat |
| | aggggtcagtcc |
| SEQ ID NO: 5 | ttagcatcaagtcctcaagagagaaaggg |
| | aatccaagaatccaaggtagtttggccaa |
| SEQ ID NO: 6 | gatcggtctgaaactcttgatattattgg |
| | ccctaatgatttttgggataaaaatcataaaagt |
| | agagttgatgaatttaataaaagaagc |
| SEQ ID NO: 7 | agaagaggcaccattcaccaagatagcaaaagat |
| | gacataaaaatacataccctaatccatttcctcc |
| | acttctctccaaaccaagtctc |
| SEQ ID NO: 8 | tgacaataggtgagatggaagcaaaaaat |
| | atatatctaccaaagatctggcacacatggcatg |
| | tctaaagcttgagctggc |
| SEQ ID NO: 9 | ctttctctttcaggtatcagtcaaaaagttctct |
| | ctaaacctacctcaaatcacttccctagcgttt |
| SEQ ID NO: 10 | aagatcacgcaggaaagagacctggtagaagaag |
| | agcccgcggggagaggtatccgagaggaagaa |
| SEQ ID NO: 11 | caatggaagggactagaagatagtaaagaccacc |
| | tggaaagatactcggtatttaaagtagcagttcc |
| | ctcatataaac |
| SEQ ID NO: 12 | catgcatgccaatatcga |
| SEQ ID NO: 13 | atgaactaacgtaattgtttagttat |
| SEQ ID NO: 14 | ttaatgagaagaccatttgtcaag |
| SEQ ID NO: 15 | gcatctctcttgttaatgtaatctt |
| SEQ ID NO: 16 | catcttgtatcataggttgtgc |
| SEQ ID NO: 17 | acctgaaatgttacttatataatttgc |
| SEQ ID NO: 18 | taggtataaatggtgtaggtaaatca |
| SEQ ID NO: 19 | ggactgacccctatttatcatc |
| SEQ ID NO: 20 | ttagcatcaagtcctcaagag |
| SEQ ID NO: 21 | ttggccaaactaccttgg |
| SEQ ID NO: 22 | gatcggtctgaaactcttga |
| SEQ ID NO: 23 | gcttcttttattaaattcatcaactct |
| SEQ ID NO: 24 | agaagaggcaccattcac |
| SEQ ID NO: 25 | gagacttggtttggagagaa |
| SEQ ID NO: 26 | tgacaataggtgagatggaag |
| SEQ ID NO: 27 | gccagctcaagctttaga |
| SEQ ID NO: 28 | ctttctctttcaggtatcagtca |
| SEQ ID NO: 29 | aaacgctagggaagtgattt |
| SEQ ID NO: 30 | aagatcacgcaggaaagag |
| SEQ ID NO: 31 | tttcttcctctcggatacct |
| SEQ ID NO: 32 | caatggaagggactagaagatag |
| SEQ ID NO: 33 | gtttatatgagggaactgctact |

1. A molecular marker for identifying the fruiting characteristics of hermaphroditic papayas, wherein the molecular markers are selected from the nucleotide sequence groups SEQ ID NO: 1 to 11.

2. A method for identifying the fruiting characteristics of hermaphroditic papayas, comprising the steps of:
obtaining genomic DNA extracted from hermaphroditic papayas under test;
analyzing whether the genomic DNA comprises any nucleotide sequence of the following group:
(i) SEQ ID NO: 3, SEQ ID NO: 5, SEQ ID NO: 10, SEQ ID NO: 11;
(ii) SEQ ID NO: 9;
wherein the existence of at least one nucleotide sequence of the group (i) indicates the fruiting characteristic of the hermaphroditic papayas is unstable;
wherein the existence of at least one nucleotide sequence of the group (ii) indicates the fruiting characteristic of the hermaphroditic papayas is stable;
wherein the unstable fruiting characteristic is lacking fruiting during fruiting period or fruiting of malformed fruits;
wherein the stable fruiting characteristics is fruiting performance without lacking fruiting during fruiting period nor fruiting of malformed fruits.

3. The method of claim 2, wherein the analysis is a high resolution melting analysis;
the high resolution melting analysis uses at least one primer pair of the following group:
(a) primer pair for detecting SEQ ID NO: 3: SEQ ID NO: 16 and SEQ ID NO: 17;
(b) primer pair for detecting SEQ ID NO: 5: SEQ ID NO: 20 and SEQ ID NO: 21;
(c) primer pair for detecting SEQ ID NO: 10: SEQ ID NO: 30 and SEQ ID NO: 31;
(d) primer pair for detecting SEQ ID NO: 11: SEQ ID NO: 32 and SEQ ID NO: 33,
(e) primer pair for detecting SEQ ID NO: 9: SEQ ID NO: 28 and SEQ ID NO: 29.

4. A method for identifying the fruiting characteristics of hermaphroditic papayas, comprising the steps of:
obtaining genomic DNA extracted from hermaphroditic papayas under test;
analyzing whether the genomic DNA comprises any homozygous allele consisting of the nucleotide sequence of the following group:
(i) SEQ ID NO: 1, SEQ ID NO: 2, SEQ ID NO: 4;
(ii) SEQ ID NO: 6, SEQ ID NO: 7, SEQ ID NO: 8;
wherein the existence of at least one homozygous allele consisting of the nucleotide sequences of (i) indicates the fruiting characteristic of the hermaphroditic papayas is unstable;
wherein the existence of at least one homozygous allele consisting of the nucleotide sequences of (ii) indicates the fruiting characteristic of the hermaphroditic papayas is stable;
wherein the unstable fruiting characteristic is lacking fruiting during fruiting period or fruiting of malformed fruits;
wherein the stable fruiting characteristics is fruiting performance without lacking fruiting during fruiting period nor fruiting of malformed fruits.

5. The method of claim 4, wherein the analysis is a high resolution melting analysis;
the high resolution melting analysis uses at least one primer pair of the following group:
(a) primer pair for detecting SEQ ID NO: 1: SEQ ID NO: 12 and SEQ ID NO: 13,
(b) primer pair for detecting SEQ ID NO: 2: SEQ ID NO: 14 and SEQ ID NO: 15,
(c) primer pair for detecting SEQ ID NO: 4: SEQ ID NO: 18 and SEQ ID NO: 19,
(d) primer pair for detecting SEQ ID NO: 6: SEQ ID NO: 22 and SEQ ID NO: 23,
(e) primer pair for detecting SEQ ID NO: 7: SEQ ID NO: 24 and SEQ ID NO: 25
(f) primer pair for detecting SEQ ID NO: 8: SEQ ID NO: 26 and SEQ ID NO: 27.
